(19) United States
(12) Patent Application Publication
(10) Pub. No.: US 2006/0135801 A1
(43) Pub. Date: Jun. 22, 2006

(54) Continuous process for the neutralization of surfactant acid precursors

(75) Inventor: Lucas Goovaerts, Haacht (BE)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 11/304,234

(22) Filed: Dec. 15, 2005

(30) Foreign Application Priority Data
Dec. 20, 2004 (EP) 04 078 430.8

(51) Int. Cl. C07C 51/00

(52) U.S. Cl. 554/124

(57) ABSTRACT

This invention provides a continuous process for the preparation of a surfactant, the process comprises the step of mixing a first component comprising a surfactant acid precursor with a second component comprising at least a molar equivalent amount of a neutralizing agent by using one or more static mixers characterized in that the neutralizing agent is added in one proportion.

### FIELD OF THE INVENTION

The present invention relates to a process for the preparation of a surfactant. More particularly, it relates to a continuous process for the preparation of a surfactant, prepared by neutralization of a first component comprising a surfactant acid precursor with a second component comprising at least a molar equivalent amount of a neutralizing agent by using one or more static mixers.

### BACKGROUND OF THE INVENTION

In the manufacture of surfactants, the surfactants are often manufactured via and supplied in their acid form. There are several reasons for this, including the fact that certain anionic surfactants, for example linear alkylbenzene sulphonates, are much easier to handle, store and transport in their acid form as compared with the neutralized form. The anionic surfactant acid precursors are then converted into their corresponding surfactant salts by neutralization with either aqueous or dry neutralizing agents.

One of the most common pieces of plant set up for carrying out neutralization of anionic surfactant acid precursors is a loop reactor. The anionic surfactant acid precursor, neutralizing agent and other diluents/buffers are injected into the loop reactor, usually at a common point, and blended by an in-line dynamic mixer present in the loop. The heat of neutralization is typically removed by a pipe bundle heat exchanger in the loop.

An inherent problem with neutralization reactions is how to deal with the large amount of heat generated. Overheating (i.e. “hot-spots”) and the long residence time can lead to discoloration of the product. Loop reactors address the problem of overheating by only removing a small fraction of the product flow, for example 5-10%, from the loop, whilst the recirculating mixture, generally in the form of a paste, acts as a heat sink, preventing a large rise in temperature at the reaction zone of the loop. This method of operation means that neutralization in a loop reactor is a highly inefficient process.

Many fully neutralized anionic surfactants tend to become highly viscous pastes which are difficult to handle. For this reason, neutralization is very often carried out in the presence of other liquid detergent components such as nonionic surfactants. However, there is a problem with discoloration of the anionic/nonionic surfactant mixture as a result of the anionic surfactant acid precursor reacting with the nonionic surfactant. It is therefore desirable that the time over which the anionic surfactant acid precursor, prior to neutralization, is in contact with the nonionic surfactant is short. The very design and operation of neutralization loop reactors means that any nonionic surfactant has multiple exposures to anionic surfactant acid precursors multiple huies as it passes around the loop multiple times.

Finally, the start-up (i.e. up to the point where a “steady-state” recirculation is achieved) and the shut-down procedures for neutralization in a loop reactor are long and time-consuming, with the material being produced during these procedures being outside of the desired specification for the neutralized product.

In order to overcome most of these disadvantages, WO 01/79 412 (Unilever, published Oct. 25, 2001) recently suggested a process for neutralizing an anionic surfactant acid precursor, in particular in the presence of a nonionic surfactant, which: (i) does not involve a recirculation loop; (ii) is relatively quick; (iii) inhibits the generation of hot-spots; (iv) is more efficient in terms of start-up and shut-down; (v) avoids the production of outside of specification material at start-up and shut-down, and (vi) ensures full neutralization of the anionic surfactant acid precursor.

In such a process, a fluid detergent product comprising an anionic surfactant can be prepared in a continuous process without the need for a loop reactor by passing the anionic surfactant acid precursor through at least two mixers in series, an initial proportion of neutralizing agent being fed to the first mixer and cooled down below 100° C. followed by further neutralizing agent being fed to the subsequent mixer or mixers to complete neutralization. Unilever admits that it is essential, in order for the process to work efficiently, that the process mixture be cooled after addition of the initial portion of neutralizing agent and before further neutralizing agent is added, and, that the temperature of the mixture be maintained at a level which allows the mixture to be readily pumpable.

There is therefore a need for the development of a process for neutralizing a surfactant acid precursor wherein such process is further simplified in comparison to the process of WO 01/79 412. Such new process should allow the addition of the entire amount of neutralizing agent in one big proportion which simplifies the technical process by the removal of unnecessary multiple injection points for the neutralizing agent. A further benefit from making a full neutralized paste in one step is the significant reduction from the risk of paste hydrolysis. Indeed when making only partly neutralized paste which is not stable, the risk of hydrolysis is substantial during start up and after shut down of the process. It is further desirable to remove or, alternatively, replace the nonionic surfactant diluent by a less sensitive component so that temperature control is less critical.

### DEFINITION OF THE INVENTION

This invention provides a continuous process for the preparation of a surfactant, the process comprises the step of mixing a first component comprising a surfactant acid precursor with a second component comprising at least a molar equivalent amount of a neutralizing agent by using one or more static mixers characterized in that the neutralizing agent is added in one proportion.

In a preferred embodiment of the present invention, the process is conducted by using two static mixers.

In another preferred embodiment of the present invention, the surfactant acid precursor is an anionic surfactant acid precursor and the process is conducted in the absence of nonionic surfactants.

In just another preferred embodiment of the present invention, the process provides high concentrated surfactant pastes with an increased surfactant activity as high as at least 80%, more preferably at least 90% and most preferably of 100%.

### DETAILED DESCRIPTION OF THE INVENTION

The Process

The process of the present invention is conducted using one or more static mixers and by adding a second component comprising an at least equivalent molar amount of neutralizing agent in one proportion to a first component comprising a surfactant acid precursor.

Static mixers

Static mixers are well-known to the skilled person. They have to be capable of operating in a continuous process and of mixing fluids. Suitable mixers include static in-line mixers, for example Sulzer-type mixers. Particularly preferred are high shear static mixers, as for example, DN 50 from Sulzer comprising 12 static mixing elements, type SMX used for mixing high viscous materials.

Static mixers are particularly preferred over dynamic mixers for the process of the present invention, as static mixers require lower capital investment. This is especially true for multi-stage high shear dynamic mixers and positive displacement pumps, which are much more expensive than static mixers used for the process of the present invention.

The fact that the present invention is a continuous process without any loop further reduces costs because less pipelines are needed and the retention time as well as start-up time are much shorter in comparison to loop processes.

The first component comprising the surfactant acid precursor is fed to the first of one or more static mixers together with a second component comprising an at least a molar equivalent amount of a neutralizing agent. The total amount of neutralizing agent needed to neutralize all surfactant acid precursor is added in one proportion. The first component and the second component can be fed separately into the first of one or more static mixers or alternatively can be brought into contact with each other prior to the first of one or more static mixers. In the case of the latter arrangement, the components should only be brought together at a position relatively close, in terms of time, to the first of one or more static mixers. Preferably the time between the two components being brought together and the combined components entering the first of one or more static mixers should be less than 3 minutes, preferably less than 1 minute.

When the combined components are leaving the first of one or more static mixers, the acid surfactant precursor is at least partially neutralized. In a preferred embodiment of the present invention, the acid surfactant precursor is fully neutralized after leaving the first of one or more static mixers.

In another preferred embodiment of the present invention, two static mixers are used. In such process set up, it is preferred that the two static mixers are in series and that there is an additional liquid injection point located between the two static mixers in series. Such additional liquid injection point can be used for the addition of other detergency components, or, for the addition of a diluent. Such diluent can be selected from various compounds and include inorganic solvents, such as water. In a preferred embodiment of the present invention, the process is conducted in the absence of nonionic surfactants.

At the very minimum, the process requires a first component comprising a surfactant acid precursor and a second component comprising a neutralizing agent as starting materials, which are of course stored in separate vessels. However, the surfactant can also contain other components. Such additional components are preferably stored separately from the surfactant acid precursor, neutralizing agent and each other. This allows a greater variety of surfactants to be prepared from the same starting materials.

Preferably, the surfactant acid precursor, neutralizing agent and any additional component can be fed from their respective storage vessels into the process independently of each other. Additional components can be fed into the process at any appropriate stage, e.g. into the first or second component, the combined components and/or a static mixer or via the additional liquid injection point representing a preferred embodiment of the present invention.

Although the various components may be fed into the process by means of gravity, it is preferred, in the case of components which are pumpable, that a pump device be used, preferably a positive displacement pump. Suitable pumps for this purpose include, for example, gear pumps and diaphragm pumps.

When the first and second components contain other component(s) in addition to the surfactant acid precursor and to the neutralizing agent, the various components are preferably brought together and mixed with the surfactant acid precursor in an additional process step preceding the first static mixer. Suitable mixers for such additional process steps include those described for the static mixers (supra) and also include dynamic in-line mixers, for example rotor-stator dynamic mixers.

Alternatively, if the constituents permit, it may be possible to premix two or more components (e.g. as a batch) and feed the premixture from a single storage vessel into the process.

The one or more static mixers are typically connected via appropriate pipelines each with each other and also with appropriate storage vessels for either the starting materials as well as for the resulting surfactant. In order to facilitate the passage of the first and second component along the pipelines and through the one or more static mixers, pumps may are used. Static mixers, by definition, do not have any moving parts, so that they do not provide a pumping action in addition to a mixing action in contrast to, e.g., rotor-stator dynamic in-line mixers.

Therefore, the pumping action imparted on the system by the pumps used to deliver the first and second components to the one or more static mixers may be sufficient for the process to operate. Alternatively, additional pumps can be incorporated along the pipelines.

Neutralization

A second component comprising an at least molar equivalent of a neutralizing agent is added to a first component comprising a surfactant acid precursor by using one or more static mixers. It is important that at least a molar equivalent of the second component is added to ensure complete neutralization of the surfactant acid precursor. If desired, a stoichiometric excess of neutralizing agent may be employed to ensure complete neutralization. For example, the process of the present invention may be conducted wherein the molar ratio between the surfactant acid precursor and neutralizing agent is from 1:1 to 1:10, preferably from 1:1 to 1:5, more preferably from 1:1 to 1:1.5 and most preferably from 1:1 to 1.05. If any other acids are present, such as for example fatty acids that require neutralization, the amount of neutralizing agent should be adjusted accordingly.

Retention Time

The period of time from first contacting neutralizing agent with the surfactant acid precursor exiting the final static mixer is herein referred to as the “retention time”. This can be measured for example by dividing the plant throughput by the plant volume. The retention time for preparation of a fully neutralized and good quality (i.e. low levels of decomposition etc.) surfactant can dependent amongst other things on temperature control, plant set up and equipment used. Typically, the retention time is less than 10 minutes. Preferably, it is less than 5 minutes, more preferably less than 3 minutes and most preferably less than 1 minute.

Temperature Control

The combined components and the neutralized surfactant exiting the final static mixer can be maintained at a temperature above the pumpable temperature at all times during the process. The “pumpable temperature” as herein defined is the temperature at which a fluid not exhibits a viscosity of 30 Pa.s at 50 s⁻¹. In other words, fluids are considered readily pumpable if they have a viscosity of no greater than 30 Pa.s at a shear rate of 50 s⁻¹ at the temperature of pumping. Fluids of higher viscosity may still in principle be pumpable, but an upper limit of 30 Pa.s at a shear rate of 50 s⁻¹ is used herein to indicate easy pumpability. The viscosity can be measured, for example, using a Haake VT500 rotational viscometer. The viscosity measurement may be carried out as follows: A SV2P measuring cell is connected to a thermostatic water bath with a cooling unit. The bob of the measuring cell rotates at a shear rate of 50 s⁻¹. The fluid, which may be in a solid form at ambient temperature, is heated in a microwave to 95° C. and poured into the sample cup. After conditioning for 5 minutes at 98° C., the sample is cooled at a rate of +/−1° C. per minute. The temperature at which a viscosity of 30 Pa.s is observed, is recorded as the “pumpable temperature”.

Therefore, it may be useful to monitor and if necessary control the temperature and thus the viscosity of each of the two components as well as of the combined components whilst the process is in operation to ensure they are both pumpable. Furthermore, it is preferred that any other components which can be incorporated into the process are maintained at a temperature above their respective pumpable temperatures when the process is in operation. Of course this does not apply in the case of any components which are solids or which are not pumpable. As components (or precursors thereof) are mixed in the process, the pumpable temperature can increase dramatically. For example, neutralized anionic surfactants are often viscous pastes whereas anionic surfactant acid precursors are often readily pumpable liquids. Thus as neutralizing agent is added to the first component, there is typically an increase in the pumpable temperature. However, the neutralization reaction generates its own heat so it is not necessarily a requirement that the process stream be heated at this point in the process. In fact, in a preferred embodiment the neutralization process can be actively cooled after addition the neutralizing agent. This can be achieved either by additional cooling means or by the addition of a diluent. Such diluent can be selected from various compounds and include inorganic solvents, such as water. In a preferred embodiment of the present invention, the process is conducted in the absence of nonionic surfactants.

In a typical embodiment, the temperature of the uncombined first and second component is maintained below 100° C., preferably below 80° C. and more preferably below 60° C. The temperature of the combined first and second component is typically maintained above 100° C., preferably above 120° C., more preferably above 140° C. and most preferably above 160° C., but below 250° C., preferably below 220° C., more preferably below 200° C. and most preferably below 175° C. It can be preferred that the temperature of the separated and combined first and second components are carefully monitored and controlled if necessary by means of heating and cooling means. It is also possible to incorporate feedback control systems into the process. For example, a temperature measuring device downstream of a cooling device can feedback readings to the cooling device and vary the level of cooling so as to maintain the temperature within a predetermined range. Of course, once the surfactant has exited the final static mixer (i.e. the process has been completed) it can be allowed to cool to a temperature below its pumpable temperature. Indeed, the use of a “structured blend” (see below) which is pumpable at elevated temperatures and yet solid at lower temperatures is a preferred embodiment of this invention. However, even when the surfactant is of the structured blend type, it is preferred to maintain the surfactant at a temperature above its pumpable temperature so it can be applied directly as, for example, a liquid binder in a granulation process without the need for reheating.

Pressure control

The pressure inside the static mixer may raise due to the component flow-through. Typically the pressure inside the static mixers is higher than atmospheric pressure. It is particularly preferred that the pressure inside the static mixers is higher than atmospheric pressure as steam formation is thereby avoided. In a preferred embodiment of the present invention, the pressure inside the static mixers is higher than 200.000 Pa, more preferably higher than 300.000 Pa, even more preferably higher than 450.000 Pa and most preferably higher than 600.000 Pa. Typically the pressure inside the static mixers is lower than 1.500.000 Pa, preferably lower than 1.000.000 Pa, more preferably lower than 900.000 Pa, even more preferably lower than 800.000 Pa and most preferably lower than 750.000 Pa. The pressure can be measured by means of a simple pressure gauge.

Heating Means

Heating means may be positioned anywhere in the process to ensure a particular fluid component or mixture is above its pumpable temperature. Suitable heating means will be apparent to the skilled person.

Cooling Means

Suitable cooling means will be well known to the skilled person and include, for example, pipe bundle heat exchangers, plate heat exchangers and frame heat exchangers.

It can be desired that at least one cooling means is provided through which the combined first and second component pass prior to the addition of any further component and/or further to the pass through of any further static mixer. The cooling means may be positioned before, at or after the first static mixer as is appropriate. Preferably, it is positioned around the first static mixer.

Further cooling means may be positioned anywhere in the process as is appropriate to control the temperature. It is particularly preferred to position further cooling means in a position where the combined first and second component are likely to be particularly hot, e.g. due to exothermic heat generated by neutralization. Thus, it is preferred that a cooling means be positioned downstream of the point of addition of the second component and preferably upstream of the point of addition of any further component. Suitably, cooling means are positioned after and around static mixer(s) where either neutralizing agent has been fed into that static mixer or to the combined first and second components entering that static mixer.

The entire neutralization process is continuous. Thus, as will be apparent to the skilled person, the static mixers, cooling means and, where appropriate, heating means should be suitable for a continuous process.

The process of this invention has been found to produce surfactant of excellent color. In other words, there is little or no discoloration as a result of the process. Furthermore, the process of the invention is highly efficient in terms of the neutralization reaction, and little or no unreacted acid is found to be present in the surfactant.

The start-up procedure is far simpler than that involved in a loop recirculation system as there is no need to wait for a steady state to develop. In addition, shut-down procedure is much simpler, as there amount of material in the system when it is in operation is far less than that in a loop system. The material produced during start-up and shut-down is also substantially of the required specification.

The Surfactant

As used herein, the term “surfactant” and/or the term “surfactant acid precursor” encompasses blends of different surfactant molecules and/or surfactant acid precursor molecules.

This invention provides a process in which a first component comprising a surfactant acid precursor is mixed with a second component comprising at least a molar equivalent amount of a neutralizing agent to fully neutralize the surfactant acid precursor resulting in the formation of a surfactant.

In a preferred embodiment of the present invention, the surfactant contains an anionic surfactant. Suitable anionic surfactants are well-know to those skilled in the art. Examples suitable for incorporation into the first component include alkylbenzene sulphonates, particularly linear alkylbenzene sulphonates having an alkyl chain length of C8-C15; primary and secondary alkyl sulphates, particularly C12-C15 primary alkyl sulphates; alkyl ether sulphates; olefin sulphonates; alkyl xylene sulphonates; dialkyl sulphosuccinates; and fatty acid ester sulphonates. Sodium salts are generally preferred. It is an essential element of the process of this invention that at least a portion, and preferably a substantial portion, of the anionic surfactant in the surfactant be formed via neutralization of an anionic surfactant acid precursor. Preferably, at least 50 wt %, more preferably at least 75 wt %, and yet more preferably substantially all of the anionic surfactant is obtained by neutralization of anionic surfactant acid precursor.

The content of anionic surfactant in the surfactant may be as high as possible, e.g. at least 98% wt. of the surfactant, or it may be less than 95% wt., or less than 50% wt. Preferably, it is at least 10% wt., more preferably at least 25% wt., more preferably at least 50% wt. and most preferably at least 75% wt. of the surfactant.

Typically, the first component comprises at least some surfactant acid precursor, preferably (a) from 20% to 98% wt. of surfactant acid precursor and (b) from 2% to 80% wt. of a liquid carrier. More preferably, the first component comprises (a) from 50% to 95% wt. of a surfactant acid precursor and (b) from 5% to 50% wt. of a liquid carrier.

Preferably, a degree of neutralization of at least 80% wt., more preferably of at least 90% wt., and more preferably substantially all of the surfactant acid precursor to be neutralized in the process.

Suitable anionic surfactant acid precursors include, for example, linear alkyl benzene sulphonic (LAS) acids, alphaolefin sulphonic acids, internal olefin sulphonic acids, fatty acid ester sulphonic acids and combinations thereof. The process of the invention is especially useful for producing compositions comprising alkyl benzene sulphonates by reaction of the corresponding alkyl benzene sulphonic acid, for instance Dobanoic acid ex Shell.

Linear or branched primary alkyl sulphates (PAS) having 10 to 15 carbon atoms can also be used.

Some of the anionic surfactant present in the surfactant may also be incorporated by direct addition of anionic surfactant at an appropriate stage in the process. However, if the first component contains anionic surfactant (i.e. a neutral salt), it accounts for less than 50 wt %, preferably for less than 25 wt %, and more preferably less than 10 wt % of the first component.

Neutralising Agent

Surfactant is formed in situ by reaction of an appropriate acid precursor and a neutralizing agent. Such neutralizing agent is preferably selected from alkaline inorganic materials, alkaline earth inorganic materials, and mixtures thereof. In principle, any alkaline inorganic material can be used for the neutralization of the surfactant acid precursor but water-soluble alkaline inorganic materials are preferred.

In a preferred embodiment, the neutralizing agent is a liquid or solution which is pumpable.

In another preferred embodiment of the present invention, the neutralizing agent is an alkali metal hydroxide. A more preferred neutralizing agent is sodium hydroxide. The latter normally must be dosed as an aqueous solution, which inevitably incorporates some water. Moreover, the reaction of an alkali metal hydroxide and acid precursor also yields some water as a by-product. Typically, the second component comprises (a) from 20% to 98% wt. of neutralizing agent and (b) from 2% to 80% wt. of a liquid carrier. Preferably, the second component comprises (a) from 40% to 80% wt. of neutralizing agent and (b) from 20% to 60% wt. of a liquid carrier. In a even more preferred embodiment of the present invention, the second component comprises (a) from 45% to 60% wt. of neutralizing agent and (b) from 40% to 55% wt. of a liquid carrier. In the most preferred embodiment of the present invention, the second component comprises (a) from 45% to 60% wt. of sodium hydroxide, and (b) from 40% to 55% wt. of water.

Another preferred neutralizing agent is sodium carbonate, alone or in combination with one or more other water-soluble inorganic materials, for example, sodium bicarbonate or silicate.

It may be advantageous to produce a second component which is alkali. For example, a pH in the range from 8.5 to 11.5. This has the advantage of ensuring the first component is completely neutralized whilst not being of such a high level alkalinity that discoloration might occur.

Of course, the second component comprising a neutralizing agent in addition to reacting with the first component comprising a surfactant acid precursor can also neutralize other acid precursors that may be present, for example fatty acids. Thus sufficient neutralizing agent needs to be added to ensure complete neutralization of all acid precursors if this is the case.

Organic neutralizing agents may also be employed.

Moisture/Water

In a preferred embodiment the surfactant is substantially non-aqueous. That is to say, the total amount of moisture therein is not more than 35% wt. of the surfactant, more preferably not more than 22% wt., most preferably not more than 18% wt. However, if desired, a controlled amount of water may be added to facilitate neutralization. Typically, the water may be added in amounts of 0.5% to 20% wt. of the surfactant. Typically, from 3% to 5% wt. of the liquid binder may be water as the reaction by-product and the rest of the water present will be the solvent in which the alkaline material was dissolved. The surfactant most preferably comprises 7% wt. of water of less.

Further Optional Process Steps

In addition to the very essential neutralization step of the present invention, the process of the present invention may comprise further process steps. One example of an additional process step is flash-drying. In a preferred embodiment of the present invention, the surfactant prepared by the process of the present may be flash-dried. Flash-drying is a process step well known to the ordinary person skilled in the art.

Most Preferred Process of the Present Invention

The most preferred process of the present invention is a continuous process for the preparation of a surfactant, the process comprising the step of mixing a first component comprising an anionic surfactant acid precursor with a second component comprising a neutralizing agent selected from alkaline inorganic materials, alkaline earth inorganic materials, and mixtures thereof, wherein the molar ratio of the first to second component is from 1:1 to 1:1.5 by using one or more static mixers characterized in that the neutralizing agent is added in one proportion wherein the pressure inside the static mixer is above 300.000 Pa to attain a degree of neutralization of at least 80% and a moisture content of the surfactant of less than 20% wt.

### EXAMPLES

The following are examples of a single continuous process for the preparation of a surfactant comprising LAS.

1. HLAS acid as a first component having a temperature of 35° C. is pumped using a first positive displacement pump from a first storage vessel and is dosed continuously controlled by a first mass flow meter, to a second component containing an aqueous solution of 50% wt. of sodium hydroxide. The combined components are fed into static in-line mixer. The amount of neutralizing agent added is sufficient to completely neutralize the LAS acid of the first component. The 50% wt. solution of sodium hydroxide is dosed using a second positive displacement pump controlled by a second mass flow meter. At the point in time where the combined components enter the static mixer and start reacting, the temperature rises up to 175° C. and the pressure at the entrance of the static mixer is 900.000 Pa. When exiting the static mixer, the temperature is 140° C. and the pressure is 450.000 Pa. The blend is of good color and fully neutralized.

2. HLAS acid as a first component having a temperature of 55° C. is pumped using a positive displacement pump from a storage vessel and is dosed continuously to a second component containing an aqueous solution of 50% wt. of sodium hydroxide. The combined components are fed into a first static in-line mixer. The amount of neutralizing agent added is sufficient to completely neutralize the LAS acid of the first component. The 50% wt. solution of sodium hydroxide is dosed using a positive displacement pump controlled by a mass flow meter. At the point in time where the combined components enter the first static mixer, the temperature rises up to 175° C. and the pressure at the entrance of the first static mixer is 900.000 Pa. When exiting the first static mixer, the temperature is 150° C. and the pressure is 700.000 Pa 50 s⁻¹. The blend is now entering a second static mixer prior to which an additional liquid injection point is placed by which additional water is added to the process. The diluted blend is then passed through a second static mixer. When exiting the second static mixer, the temperature is 130° C. and the pressure is 450.000 Pa. The blend is of good color and fully neutralized.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A continuous process for the preparation of a surfactant, the process comprising the step of mixing a first component comprising a surfactant acid precursor with a second component comprising at least a molar equivalent amount of a neutralizing agent by using one or more static mixers wherein in that the neutralizing agent is added in one proportion.

2. The process according to claim 1 by using two static mixers.

3. The process according to claim 2 wherein the two static mixers are in series and wherein there is a liquid injection point located between the two static mixers in series.

4. The process according to claim 1 wherein the surfactant acid precursor is an anionic surfactant acid precursor and wherein the process is conducted in the absence of nonionic surfactants.

5. The process according claim 1 wherein the neutralizing agent is selected from the group consisting of alkaline inorganic materials, alkaline earth inorganic materials, and mixtures thereof.

6. The process according to claim 1 wherein a degree of neutralization of at least about 80% is attained.

7. The process according to claim 1 wherein the pressure inside the static mixer is above about 200,000 Pa.

8. The process according to claim 1 wherein the temperature inside the static mixer is above about 100° C. but lower than about 250° C.

9. The process according to claim 1 wherein the residence time of the surfactant in the static mixer is shorter than about 10 minutes.

10. The process according to claim 1 wherein the process further comprises the step of flash-drying.

11. The process according to claim 1 wherein the process further comprises the step of flash-drying the surfactant.

12. The process according to claim 1 wherein the first component comprises (a) from about 20% to about 98% wt. of surfactant acid precursor and (b) from about 2% to about 80% wt. of a liquid carrier.

13. The process according to claim 1 wherein the second component comprises (a) from about 20% to about 98% wt. of neutralizing agent and (b) from about 2% to about 80% wt. of a liquid carrier.

14. The process according to claim 1 wherein the molar ratio between the surfactant acid precursor and neutralizing agent is from about 1:1 to about 1:10.

15. The process of according to claim 1 wherein the moisture content of the surfactant is less than about 35% wt.

16. A continuous process for the preparation of a surfactant, the process comprising the step of mixing a first component comprising an anionic surfactant acid precursor with a second component comprising a neutralizing agent selected from alkaline inorganic materials, alkaline earth inorganic materials, and mixtures thereof, wherein the molar ratio of the first to second component is from about 1:1 to about 1:1.5 by using one or more static mixers wherein the neutralizing agent is added in one proportion and wherein the pressure inside the static mixer is above about 300,000 Pa to attain a degree of neutralization of at least about 80% and a moisture content of the surfactant of less than about 20% wt.
